(19) United States
(12) Patent Application Publication
(10) Pub. No.: US 2026/0142041 A1
(43) Pub. Date: May 21, 2026

(54) SYSTEMS AND METHODS FOR DRUG INTERACTION ANALYSIS

(71) Applicant: Blue Genes Lab, LLC, Scottsdale, AZ (US)

(72) Inventors: Bill Massey, Scottsdale, AZ (US); Christopher Diaz, Scottsdale, AZ (US); Nocholas Glimcher, Scottsdale, AZ (US)

(21) Appl. No.: 19/235,420

(22) Filed: Jun. 11, 2025

Related U.S. Application Data
(63) Continuation of application No. 19/117,365.
(60) Provisional application No. 63/411,911, filed on Sep. 30, 2022.
(60) Provisional application No. 63/464,149, filed on May 4, 2023.

(51) Int. Cl. G16H 70/40; G16B 20/20; G16H 15/00

(52) U.S. Cl. CPC G16H 70/40; G16B 20/20; G16H 15/00

(57) ABSTRACT

The present disclosure is directed to systems and methods for analyzing drug-drug and gene-drug interactions for a user and providing recommendations accordingly. The systems and methods can utilize a database comprising a list of genes are associated with a plurality of drugs based on clinical relevance of the gene to the actions of the plurality of drugs. The systems and methods can include recommending alternative drugs to users based on any identified problematic drug-drug or gene-drug interactions.

### CROSS-REFERENCE TO RELATED APPLICATIONS

This application is a continuation of U.S. application Ser. No. 19/117,365, filed on Mar. 31, 2025 entitled “SYSTEMS AND METHODS FOR DRUG INTERACTION ANALYSIS,” which is a U.S. national phase filing under 35 U.S.C. § 371 of PCT/US2023/034534 filed on Oct. 5, 2023, which claims priority to U.S. Provisional Patent Application No. 63/464,149 filed on May 4, 2023 and U.S. Provisional Patent Application No. 63/411,911 filed on Oct. 6, 2022, the disclosures of which are incorporated herein by reference.

### BACKGROUND

Two of the major determinants of whether a drug will have the intended therapeutic effect or produce an adverse drug reaction are the patient's genetic make-up and the potential for two concomitantly administered drugs to interact (drug-gene and drug-drug interactions, respectively). Currently, drugs are generally prescribed to patients without performing detailed genetic analyses. Further, if patients do not provide their healthcare providers with a complete and accurate list of all of the drugs that they are taking (either intentionally or unintentionally), then healthcare providers may prescribe a drug that has a negative interaction with another drug. This can cause negative health outcomes for patients and inefficiencies related to drug prescribing, generally referred to as “trial-and-error” prescribing. Therefore, there is a need in the art for a system that optimizes the mix of drugs a patient is prescribed according to genetic make-up and drug-to-drug interactions. Absent such information, one can never be assured that the drugs that they are taking are in fact completely correct for a particular patient. Some current commercially available databases have some information on drug-drug interactions and some information on the drugs themselves (e.g., major metabolite or the enzyme that forms that metabolite), but such commercial databases do not provide all of the information needed to make proper assessments regarding potential drug-drug and/or gene-drug interactions. Therefore, there is a need in the prior art for systems and methods that will fully analyze all drug-drug and gene-drug interactions in order to provide better clinical outcomes and more savings throughout the healthcare industry (whether by avoiding negative health outcomes for patients or by minimizing prescribing inefficiencies).

Pharmacogenomic testing identifies mutations or variants in genes that may affect whether a particular medication would be an effective treatment for the individual or whether the tested individual would suffer from side effects to the medication. Current pharmacogenetic test reports list each gene, genotype, and phenotype separately, and include a list of drugs affected by each gene. Therefore, the onus is placed on the medical provider to properly digest and interpret the myriad information in the pharmacogenetic test report to provide the best medication recommendation to the patient. Understandably, many providers find the pharmacogenetic test reports confusing and have difficulty incorporating the testing information into their usual practice of medicine due to (i) a general lack of expertise in interpreting pharmacogenetic data, (ii) time constraints caused by their daily patient volumes, (iii) challenges in integrating synthesizing data from multiple sections of the pharmacogenetics reports related to different genes and understanding the significance of the different sections for a particular drug, and (iv) the fact that the currently generated pharmacogenetics reports are static and, thus, have limited utility following any changes to the patient's drug regimen. Therefore, there is a need in the prior art for systems adapted to report results and recommendations from analyzed drug-drug and gene-drug interactions in a more understandable and dynamic manner.

### SUMMARY

The present disclosure is directed to systems and methods for analyzing drug-drug and/or drug-gene interactions for a user. In some embodiments, the systems and methods include providing users with recommendations regarding any identified drug interactions. Described herein is a clinical decision support tool that allows physicians and patients to check their medications for suitability with each other (drug-drug interactions) and suitability with the patient's genetics (gene-drug interactions) in an integrated manner. The system flags potential interactions and provides alternative medications that are genetically suitable for the patient. This allows the physician to prescribe a medication regimen that is optimal for a particular patient.

In some embodiments, the present disclosure is directed to a method for analyzing drug interactions for a user, the method comprising: receiving genetic test results for the patient and a drug taken by the user, wherein the drug is in a drug category; determining whether the drug is present in an internal database, wherein the internal database comprises a list of genes associated with a plurality of drugs based on clinical relevance of the gene to the actions of the plurality of drugs; in response to determining that the drug is not present in the internal database: searching an external database for a major metabolite and an enzyme responsible for forming the major metabolite, wherein the major metabolite and the enzyme are associated with the drug, and determining whether there is a mutation in a gene associated with the enzyme that pharmacokinetically impacts the drug based on the genetic tests results; in response to determining that the drug is present in the internal database: determining a clinically relevant enzyme associated with the drug from the internal database, and determining whether there is a mutation in the clinically relevant gene based on the genetic tests results; determining whether there is a problematic gene-drug interaction associated with the drug; in response to determining that there is the problematic gene-drug interaction, identifying an alternative drug from the drug category (either from the internal database or external database as previously described); and providing a report to the user, the report comprising the problematic gene-drug interaction and the alternative drug.

In some embodiments, the present disclosure is directed to a method for analyzing drug interactions for a user, the method comprising: receiving genetic test results and a drug or drugs (drug(s)) for the user; determining a primary metabolite for the drug(s) and a pathway for the primary metabolite(s); determining a category for the drug(s); determining a primary enzyme(s) pharmacokinetically associated with the drug(s) based at least in part on the primary metabolite(s) and the pathway; determining, based on the genetic test results, whether the user has a genetic mutation associated with the determined primary enzyme(s) that would pharmacokinetically impact the drug(s); in response to a determination that the patient has the genetic mutation, determining whether the genetic mutation has a threshold pharmacokinetic impact on the primary enzyme(s); in response to a determination that the genetic mutation has the threshold pharmacokinetic impact, determining an alternative drug(s) to the drug(s) based on the determined category for the drug(s); providing, to the user, an indication of a pharmacokinetic impact of the genetic mutation and the alternative drug(s).

In some embodiments, the present disclosure is directed to a system for analyzing drug interactions for a user, the system comprising: a processor; and a memory coupled to the processor, the memory storing instructions that, when executed by the processor, cause the system to: receive genetic test results for the patient and a drug taken by the user, wherein the drug is in a drug category, determine whether the drug is present in an internal database, wherein the internal database comprises a list of genes associated with a plurality of drugs based on clinical relevance of the gene to the actions of the plurality of drugs; in response to determining that the drug is not present in the internal database: search an external database for a major metabolite and an enzyme responsible for forming the major metabolite, wherein the major metabolite and the enzyme are associated with the drug, and determine whether there is a mutation in a gene associated with the enzyme that pharmacokinetically impacts the drug based on the genetic tests results, in response to determining that the drug is present in the internal database: determine a clinically relevant enzyme associated with the drug from the internal database, and determine whether there is a mutation in the clinically relevant gene based on the genetic tests results; determine whether there is a problematic gene-drug interaction associated with the drug; in response to determining that there is the problematic gene-drug interaction, identify an alternative drug from the drug category; and provide a report to the user, the report comprising the problematic gene-drug interaction and the alternative drug.

In some embodiments, the disclosed systems and methods provide dynamic reports that integrate pharmacogenetic test information across multiple genes relevant to an individual drug. In some embodiments, the disclosed systems and methods label drug risks in a clear manner that is more easily understood in a manner that does not require deep knowledge of pharmacogenetics, which is applicable to both patients and medical practitioners.

### FIGURES

The accompanying drawings, which are incorporated in and form a part of the specification, illustrate the embodiments of the invention and together with the written description serve to explain the principles, characteristics, and features of the invention. In the drawings:

FIG. 1 depicts a block diagram of a system for analyzing drug interactions for a user, in accordance with an embodiment of the present disclosure.

FIG. 2A depicts a flow diagram of a process for analyzing drug interactions for a user, in accordance with an embodiment of the present disclosure.

FIG. 2B depicts a flow diagram of a process for analyzing drug-gene interactions and categorizing a drug accordingly, in accordance with an embodiment of the present disclosure.

FIG. 3A depicts an illustrative graphical user interface (GUI) for inputting drugs, in accordance with an embodiment of the present disclosure.

FIG. 3B depicts an illustrative GUI for indicating drug interactions, in accordance with an embodiment of the present disclosure.

FIG. 4 depicts an illustrative internal or override table, in accordance with an embodiment.

### DETAILED DESCRIPTION

This disclosure is not limited to the particular systems, devices and methods described, as these may vary. The terminology used in the description is for the purpose of describing the particular versions or embodiments only and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

### Definitions

As used herein, the singular forms “a,” “an,” and “the” include plural references, unless the context clearly dictates otherwise. Thus, for example, reference to a “pharmaceutical” is a reference to one or more pharmaceuticals and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

As used herein, terms “user,” “subject,” or “patient” can be used interchangeably.

As used herein, the terms “drug,” “medication,” or “pharmaceutical composition” refer to a composition comprising an active pharmaceutical ingredient to be delivered to a subject, for example, for therapeutic, prophylactic, diagnostic, preventative, or prognostic effect.

As used herein, the term “active pharmaceutical ingredient” or “API” refers a substance in a pharmaceutical composition that provides a desired effect, for example, a therapeutic, prophylactic, diagnostic, preventative, or prognostic effect. In various embodiments, the active pharmaceutical ingredient can be any of a variety of substances known in the art, for example, a small molecule, a polypeptide mimetic, a biologic, an antisense RNA, a small interfering RNA (siRNA), and so on.

As used herein, “analyzing drug interactions” refers to assessing the impact of a patient's genetics on the metabolism of a drug, i.e., analyzing how a drug is metabolized by a patient, wherein the determination is based on the genetics of the patient.

As used herein, “genetic test results” refers to results from one or more of molecular tests (e.g., targeted single variant tests, single gene tests, gene panels, and/or whole genome sequencing), chromosomal tests, gene expression tests, or biochemical tests for identifying genotypes and/or phenotypes of genes. In particular embodiments, the genetic test results are for genes that are relevant for drug response as described herein.

As used herein, “drug category” refers to classifications for drugs based on therapeutic effect, mechanism of action, or other collective characteristics. Drug categories could include, for example, opioids, analgesics, beta blockers, and antipsychotics.

As used herein, “clinical relevance” refers to whether the gene directly or indirectly impacts the drug's metabolism. Relevant genes for various types of drugs would be known by the skilled person by, for example, consulting available scientific literature, FDA labeling, and other accepted sources of drug information. In one illustrative embodiment, a mutation in a gene that enhances or reduces a particular drug's metabolism by at least a threshold amount (e.g., 50% or greater) could be considered clinically relevant for the drug.

As used herein, “major metabolite” refers to the product predominantly produced by action of the enzyme on the drug.

As used herein, “associated with the drug” or “pharmacokinetically associated with the drug” refers to an enzyme that interacts with the drug or a protein that otherwise affects the response to the drug (e.g., by interacting with a receptor or membrane protein, such as a transporter), either directly or indirectly, to produce the major metabolite (e.g., by using the drug as a substrate) or otherwise influence the therapeutic response to the drug.

As used herein, “problematic,” particularly within the context of a problematic gene-drug interaction, refers to whether the drug's action is affected by one or more genetic mutations of the user. This effect may be to reduce or increase the action of the drug. Increasing or reducing the action of the drug may lead to adverse effects for the patient.

As used herein, “threshold pharmacokinetic impact” refers to any factor that reduces or enhances drug metabolism by a threshold amount (e.g., 50% or greater)

As used herein, “determining whether there is a problematic gene-drug interaction associated with the drug” refers to determining whether the genetics of the user (i.e., mutations in a gene associated with a protein which acts either directly or indirectly on the drug) impact metabolism of or response to the drug. Problematic gene-drug interactions can be calculated by, for example, examining the scientific data regarding metabolism or response to the drug and whether mutation of the protein encoded by the gene can affect the response or metabolism of the drug in a clinically relevant manner.

As used herein, “internal database” refers to a database including one or more entries, wherein each database entry could include a variety of different information, such as the clinically relevant genes or genetic mutations for specific drugs, the degree to which the listed genes affect drug metabolism or drug response, and other gene-drug interactions.

As used herein, “external database” refers to a database including one or more entries, wherein each database entry could include a variety of different information, such as the drug's major metabolic pathways, the classification or category of the drug, and drug-drug interactions. A suitable external database could include, for example, the DrugBank database, which is accessible at https://go.drugbank.com.

### Drug Interaction Analysis System

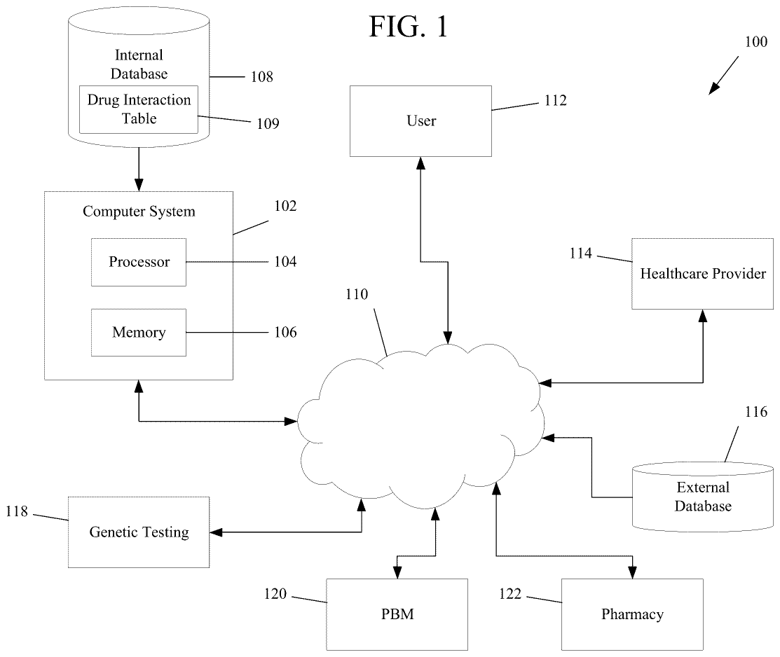

The present disclosure is directed to systems and methods for analyzing medications for a patient, including analyzing drug-drug and gene-drug interactions. Further, the described systems and methods can recommend alternative medications to the patient and/or a third party (e.g., a healthcare provider) in the event that an alternative medication could be warranted for the patient. FIG. 1 illustrates a system 100 including a computer system 102 that can be accessed by a user 112 and/or a healthcare provider 114 via a network 110 (e.g., the Internet). Further, the computer system 102 could store or otherwise be communicatively coupled to a database 110, which can store a variety of different information associated with drug pharmacokinetics or drug-drug interactions, as described in greater detail below. In operation, the user 112 can access the computer system 102 to input data (e.g., medications that the user is taking), review pharmacogenetic analysis results and recommendations, and take other such actions.

In one embodiment, the computer system 102 could be operated or controlled by a testing entity that is able to receive, perform, and/or order genetic testing 118 on patient-provider samples to obtain genetic data on the patient. In another embodiment, the computer system 102 could be otherwise configured to receive genetic testing 118 from an external source (e.g., via the network 110). As described in greater detail below, users' genetic data can be utilized to analyze whether any alternative medications may provide better results or may otherwise be better suited for the user given the user's genetic makeup. In one embodiment, the user's genetic test results can be obtained by laboratory testing of genetic samples taken from the user (e.g., through analysis of buccal mucosal epithelial cells taken via swabbing the inside of the check with a specially manufactured swab). The genetic test results can be in the form of the genetic diplotype (i.e., one allele from each parent), which is then converted into a phenotype (i.e., the functional impact of the diplotype on protein function). In some embodiments, it could be beneficial to consider a user's diplotype because each allele can be associated with a functional aspect of the protein for which it encodes. Accordingly, an individual's diplotype could influence how he or she metabolizes a drug or how the drug otherwise affects the individual. For example, if one allele encodes a protein that exhibits normal functionality and the second allele encodes a non-functional protein, then the blended phenotype could operate as a half-functional protein. For a gene that encodes a metabolic enzyme, the example phenotype for the individual could be categorized as an “intermediate metabolizer,” for example. In one embodiment, the genetic testing 118 could include the diplotype associated with one or more tested genes. In this embodiment, the computer system 102 could be programmed to match the received diplotype from the genetic test results with a table of previously determined functionalities associated with individual alleles and, accordingly, convert the diplotype into a predicted blended phenotype for the gene. The phenotype could then be reported as the clinically relevant result for that gene. In some embodiments, the phenotype could be assigned a corresponding indicium from one or more indicia that represent the degree of impact on protein function for the user's diplotype. For example, the indicia could represent severe, intermediate, or no impact. In one embodiment, the indicia for the assigned phenotypes could include a color (e.g., red, yellow, and green).

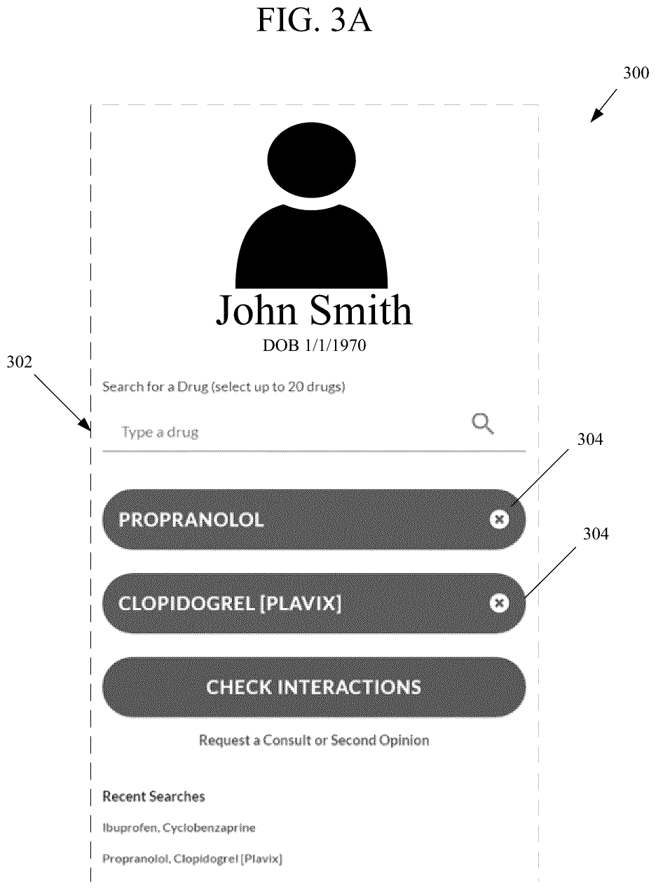

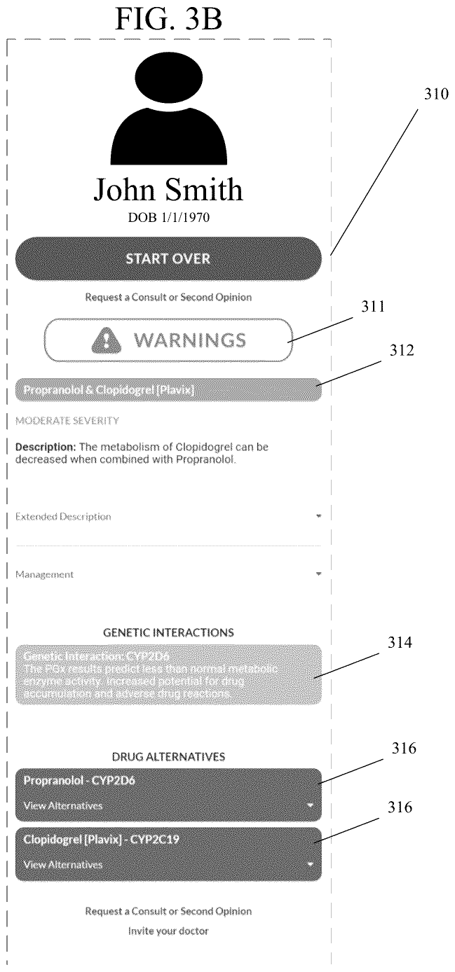

The computer system 102 can include a processor 104 and a memory 106 to execute various process or algorithms for analyzing the drugs users are taking and/or users' data to provide recommendations to the users accordingly. In some embodiments, the computer system 102 could include a server or a cloud-based computing system. In various embodiments, the computer system 102 could be configured to provide an interface (e.g., a GUI) that is accessed by and/or interacted with via a software application (e.g., run on a mobile device associated with the user 112) or a website. The interface could allow users to input the medications that they are taking, such as is shown in FIG. 3A. Further, the interface could display various recommendations or alerts to users, such as is shown in FIG. 3B.

In one embodiment, a user could manually input which drugs they are taking and/or have been prescribed. In another embodiment, the computer system 102 could be configured to obtain drugs the user is taking from the user's electronic health records (EHR) or other external source. For example, the computer system 102 could be communicably coupled to a healthcare provider computer system and have access to the user's EHR through either permission from the user or the user's healthcare provider 114 (e.g., doctor). In this embodiment, the computer system 102 could be configured to automatically retrieve the list of the drugs that the user is taking and/or has been prescribed from the user's EHR upon receipt of the appropriate permissions.

The computer system 102 can be configured to communicate with an external database 116 that provides information on drugs, such as drug-drug interactions or information on the major metabolic pathway associated with the drug (e.g., the major circulating metabolite and the metabolic enzyme responsible for its formation). The external database 116 could be accessed via a corresponding application programming interface (API), for example. The external database 116 could include the DrugBank database, for example. One issue with current drug databases, such as DrugBank, is that although they may have some information on drug-drug interactions and major metabolic pathway data for the drug (e.g., the major circulating metabolite and the metabolic enzyme responsible for its formation), the available information in such external databases 116 is not sufficient to determine whether any genetic mutations an individual may have (as determined from genetic testing) could affect the pharmacokinetics of a given drug. For example, although currently available drug information databases may include the metabolic enzyme associated with the major circulating metabolite associated with the drug, the pharmacokinetics of a given drug may be affected by other enzymes and/or other genetic mutations. Therefore, currently available databases are not sufficient to provide individuals with a complete, clear picture on whether a drug may be appropriate for them to use based on genetic testing.

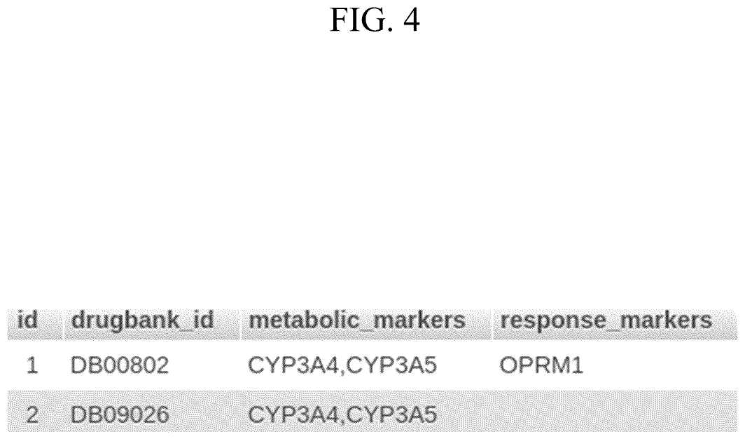

Accordingly, the system 100 described herein includes a drug interaction table 109 associated with an internal database 108 that is stored on the computer system 102 or is otherwise communicably coupled to the computer system 102. The drug interaction table 109, also referred to in some instances as an “override table,” has been developed to supplement and/or override drug information in currently commercially available drug information databases. The drug interaction table 109 sets forth drug-gene interactions for drugs for which currently available external databases 116 do not provide sufficient information to identify all possible drug-gene interactions. For each drug in the drug interaction table 109, genes are associated with a drug based on clinical relevance of the gene to the actions of the drug. Clinical relevance is determined based on experimental data (usually clinical data) showing that genetic mutations affecting the function of the protein encoded by the gene have effects that impact the drug's clinical attributes, e.g. efficacy, safety, or pharmacokinetics. In some embodiments, the drug interaction table 109 could be based on individual alleles and/or a diplotype associated with the gene or genes set forth. In one embodiment, the drug interaction table 109 is constructed in a tabular format including each drug for which the external databases 116 lack sufficient information to identify the relevant gene-drug interactions and the clinically relevant genes associated with each of the drugs. The drug interaction table 109 can further include clinically relevant metabolic genes and clinically relevant response genes. FIG. 4, for example, depicts an illustrative portion of a drug interaction table 109 indicating the metabolic markers and response markers for a pair of drugs. In this example, the drugs are indicated based on their DrugBank IDs (e.g., DB00802 corresponds to alfentanil). Accordingly, the system 100 can query DrugBank or other external databases based on the particular IDs associated with those drugs from the drug interaction table 109.

In operation, the computer system 102 receives the list of one or more drugs that a user is taking (e.g., by being manually entered via an app GUI or a website GUI) and the user's genetic testing results. For each drug taken by the user, the computer system 102 queries the drug interaction table 109 for the drug in question, retrieves the relevant genes from the metabolic and response gene data for the drug, checks the relevant genes against the patient's pharmacogenetic test results, and reports any abnormalities that present a potential drug-gene interaction (e.g., a mutation present in a gene that codes for one of the clinically relevant enzymes associated with drug). If a drug is not present in the drug interaction table 109, the computer system 102 could retrieve corresponding data from an external database 116 and determine any drug-gene interactions therefrom. The report to the user could include a description of the nature of the abnormality and how that affects either drug metabolism or drug response. As new information regarding drug-gene interactions become available (e.g., new research and/or clinical data), the drug interaction table 109 can be dynamically modified to incorporate the new information. Unlike the internal database 108, current commercially available database (e.g., DrugBank) do not consider clinical relevance, but rather simply list drug attributes (e.g., drug metabolizing enzymes or drug class). These commercially available databases can be queried in a manner that assumes certain attributes have clinical relevance (i.e., clinical relevance may not be supported by experimental data) based on general principles of pharmacology (e.g., drug metabolism and dose-response relationships), but these assumptions would not be correct for all drugs. Therefore, any system that attempted to identify possible drug-gene interactions based on this commercially available information alone would be incomplete and, thus, not provide correct results in all cases. If the drug in the external database has drug attributes for which the tested pharmacogenetic genes are relevant, the relevant gene information can be reported if abnormal.

In one particular embodiment, the system 100 can be embodied as a web-based app that can search a commercial database of drug-drug interactions (e.g., DrugBank), identify problematic drug combinations, and display any problematic drug combinations to the physician and/or the patient user, along with extended descriptions of the problematic interaction and information on how to manage them clinically. The system 100 can further identify gene-drug interactions by comparing the patient's medications to an internal database of specific drugs and the genes that code for proteins that impact either the biotransformation of the specific drugs (i.e., metabolic genes), transport of the specific drugs within the body, and/or the therapeutic response to the specific drug (i.e., response genes). The internal database can be established using clinical and/or research data on the various drugs.

Any problematic drug-drug and/or drug-gene interactions identified by the system 100 can be indicated based on phenotypes associated with abnormal function of the encoded proteins. Further, the results can be presented to the user in a variety of different formats. For example, the results can be color-coded green, yellow, or red based on the severity of the clinical impact of the genetic mutation and/or drug-drug interaction, where green can indicate normal function, yellow can indicate an increased risk of adverse effect or decreased clinical efficacy, and red can indicate an extreme risk of adverse effect or lack of clinical efficacy.

### Drug Interaction Analysis Processes

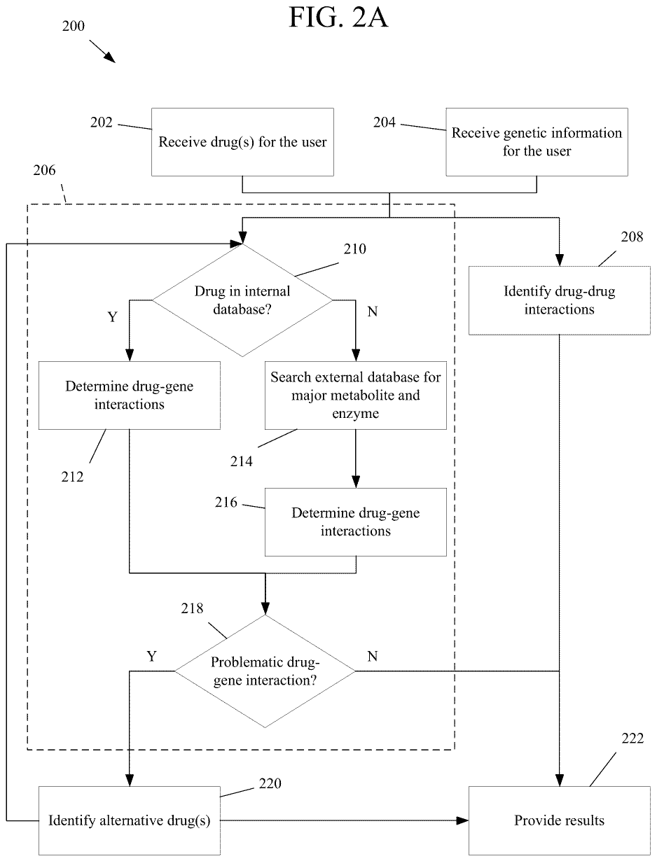

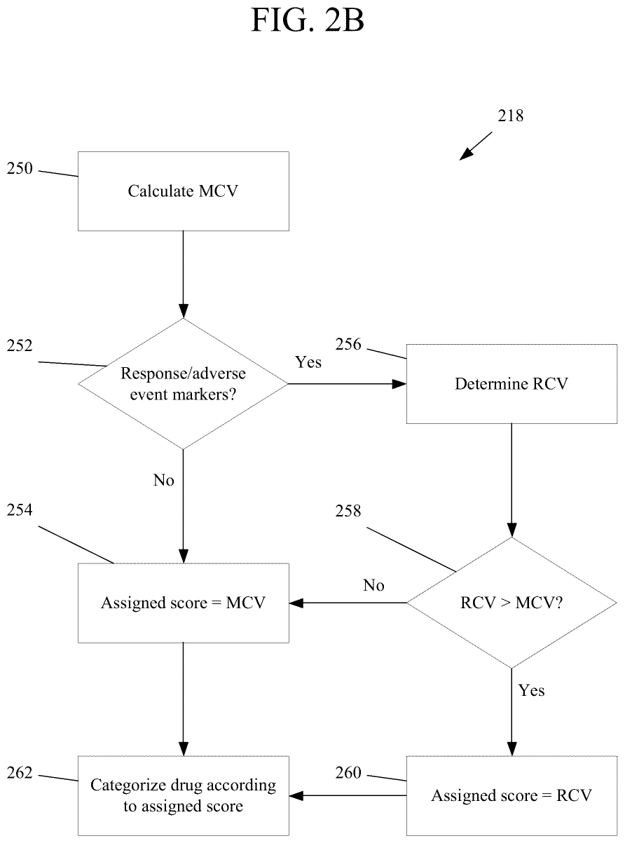

One embodiment of a process 200 for analyzing users' drug interactions is shown in FIG. 2A. In one embodiment, the process 200 can be embodied as instructions stored in a memory (e.g., the memory 106) that, when executed by a processor (e.g., the processor 104), cause the computer system 102 to perform the process 200. In various embodiments, the process 200 can be embodied as software, hardware, firmware, and various combinations thereof. In various embodiments, the process 200 can be executed by and/or between a variety of different devices or systems. For example, various combinations of steps of the process 200 could be executed by the computer system 102, the network 110, and/or device (e.g., computer, laptop, or smartphone) associated with the user 112. In various embodiments, the system 100 executing the process 200 can utilize distributed processing, parallel processing, cloud processing, and/or edge computing techniques. The process 200 is described below as being executed by the system 100; accordingly, it should be understood that the functions can be individually or collectively executed by one or multiple devices or systems.

The system 100 executing the process 200 can receive 202 one or more drugs associated with the user and receive 204 genetic information (e.g., a genetic testing results) associated with the user. In one embodiment, the user could manually input the drug(s) that he or she is taking by interacting with the computer system 102, such as by inputting the drugs in a website interface. For example, FIG. 3A illustrates a GUI 300 including a drug input field 302 in which users can manually input the drugs that they are taking and/or have been prescribed. In the particular example shown in FIG. 3A, the user has input two drugs 304 (propranolol and clopidogrel) that he is taking. In another embodiment, the computer system 102 could retrieve the user's EHR (e.g., from a database associated with the patient's healthcare provider) and determine the drugs that are being taken and/or have been prescribed to the user therefrom. In one embodiment, the system 100 could receive 204 the genetic information from a genetic test performed by the entity operating the computer system 102. In another embodiment, the system 100 could receive 204 the user's genetic information from a third party or another external source.

For each of the patient's drugs, the system 100 can identify 206 any drug-gene interactions associated with the received 202 drug list and the received 204 genetic information. In parallel with or otherwise separately from identifying 206 drug-gene interactions, the system 100 can further identify 208 any drug-drug interactions. In one embodiment, the system 100 can identify 206 drug-drug interactions utilizing commercially available databases (e.g., DrugBank).

The system 100 can identify 206 drug-gene interactions between the received 202 drug list and the received 204 genetic information for the user in a variety of different manners. In one embodiment shown in FIG. 2A, the system 100 can determine 210 whether the drug is present in the internal database 108 (i.e., the drug interaction table 109). If the system 100 determines 210 that the drug is present in the internal database 108, the system 100 can determine 212 whether there any drug-gene interactions based on the precharacterized drug-gene data in the drug interaction table 109. If the system 100 determines 210 that the drug is not present in the internal database 108, the system 100 can search 214 an external database 116 (e.g., DrugBank) for the major metabolite and the enzyme responsible for forming the major metabolite that is associated with the given drug. If a drug is not present in the internal database 108, it could be assumed that it has been determined that the information present in the external database 116 is sufficient to assess the clinical effects of pharmacogenetic data for the drug. Based on the data from the external database 116, the system 100 can identify the primary enzyme for the drug, which can in turn be utilized to determine 216 whether there any drug-gene interactions based on the user's genetic testing information and the determined primary enzyme associated with the drug. In other words, the system 100 can determine 216 whether the user has a mutation in the gene associated with the identified primary enzyme that clinically affects the drug. In this context, a genetic mutation could clinically affect the enzyme if it deactivates the enzyme, causes the enzyme to be over functional, or causes the enzyme to be under functional. For example, the CYP2C19 codes for an enzyme that is clinically relevant to clopidogrel. Clopidogrel is a prodrug which is converted to its active form by CYP2C19. If CYP2C19 has a mutation that causes the enzyme to be under functional, then there will be no clinically relevant drug effect for clopidogrel. Conversely, if CYP2C19 has a mutation that causes the enzyme to be over functional, then there will be too much drug effect and the patient's blood will be thinned too much, leading to increased risk of hemorrhage. The system 100 could determine 212, 216 whether the user has a mutation in the CYP2C19 gene or any other gene that has a clinical effect on a drug either via the precharacterized drug interaction table 109 or information obtained from an external database 116.

Accordingly, the system 100 can determine 218 whether there are any problematic drug-gene interactions. Different genetic mutations can have varying impacts on the pharmacokinetics of the medication; therefore, it may be desirable in some implementations to quantify or otherwise determine the degree of pharmacokinetic impact that the particular genetic mutation would have on the medication. In other words, it may be desirable to only recommended alternative medications in circumstances where the patient's genetic mutation would have a significant or non-trivial pharmacokinetic impact on the medication. Therefore, in one embodiment, the system 100 could determine 218 whether there are any problematic drug-gene interactions based on whether the pharmacokinetics associated with the enzyme coded by the particular gene at issue would be adversely impacted with respect to the drug by at least a threshold level or degree.

In one embodiment, the system 100 can code the analyzed drug into one or more categories depending on whether the system 100 identifies any problematic drug-gene interactions. For example, a first category could correspond to drugs for which no genetic indicators of clinical importance were identified based on the subject's genetic data, a second category could correspond to drugs for which genetic indicators were identified that warrant caution, and a third category could correspond to drugs for which genetic indicators were identified warrant extreme caution or avoidance. To aid users in quickly identifying and assessing which of the categories the analyzed drug falls into, the categories can be associated with various indicia (e.g., color) that can be presented via a graphical user interface, such as is shown in FIGS. 3A and 3B.

In embodiment, the system 100 can determine 218 whether there are any problematic drug-gene interactions by calculating a score for the drug and comparing the calculated score to various threshold values to assess whether the particular drug or set of drugs are problematic given the subject's genetic data. In embodiment, the drug score can include one or more separate calculations and/or components.

In one embodiment, determining and/or calculating the drug score could include determining and/or calculating a metabolic component value (MCV) and a response component value (RCV). The MCV can be a calculation based on the phenotypic results for the relevant metabolic genes. The RCV can be a calculation based on the expected response or adverse effects according to the phenotypes determined from the subject's genetic information. In one embodiment, MCV can be calculated for n genes according to the following equation:

$$M C V = {\sum }_{i = 0}^{n} ( {PCD}_{i} \times {I}_{i} )$$

where PCD is the phenotypic color designation for the particular gene and I is the relative importance (e.g., expressed as a weight or percentage) for the particular gene. In other words, the MCV is calculated via a weighted summation the PCDs for n genes associated with the particular drug being analyzed. Notably, the relative importance (I) for the genes associated with the drug sum to one. In one embodiment, the PCD could be a value corresponding to the assigned coding for the gene (e.g., no genetic indicators of clinical importance, genetic indicators warranting caution, or genetic indicators warranting extreme caution or avoidance). For example, the PCD value for a “green” or low risk gene for the particular drug could be one, the PCD value for a “yellow” or moderate risk gene could be five, and the PCD value for a “red” or high risk gene could be ten. In other embodiments, the PCD values could be different static values, functions based on one or more variables, and so on. In one embodiment, the relevance value could be a value assigned to each value based on its pharmacological and toxicological attributes. For example, the relevance value could be based on the overall contribution of each tested gene to the total metabolism of the drug and resultant drug metabolites, the clinical relevance of the metabolic product from each tested gene (e.g., whether active metabolites are produced, whether toxic metabolites are produced, and whether the metabolites are primary to the drug response), known pharmacogenetic-related metabolic effects (e.g., obtained from the drug label), and relevant information from the scientific literature (e.g., data from in vitro studies using human hepatocytes and clinical studies).

In one embodiment, determining and/or calculating the drug score could include determining and/or calculating a response component value (RCV). For example, the RCV calculation can be based on the PCD obtained for a response-associated gene or PCDs for a group of response-associated genes (if more than one response gene is relevant for the drug). For response genes, there is no weighting of the relative importance for the individual response genes since any deleterious mutation on any relevant response gene would make the drug not efficacious. Therefore, the highest PCD value among the relevant response genes is assigned as the RCV. So, for example, if a drug has two relevant response genes with PCD values of 1 and 5, then the RCV would be 5.

In one embodiment, the system 100 can determine 218 whether there are any problematic drug-gene interactions using a combination of the MCV and RCV. One example of a process for determining 218 whether there are any problematic drug-gene interactions given the subject's genetic information and drug(s) associated with the subject (i.e., drugs currently being taken by the subject or drugs that are to be prescribed to the subject) is shown in FIG. 2B. In this embodiment, the system 100 calculates 215 the MCV given the genetic information and drug(s) received 202, 204 for the subject, as described above. Further, the system 100 determines 252 whether any response and/or adverse event markers are present. The system 100 surveys the genetic information obtained by testing for pharmacogenetic mutations (i.e., the PGx test results) and matches it with the genes that are relevant for the drug for which the PCD is being determined. If no response and/or adverse event markers are present, then the system 100 assigns 254 the MCV as the score for the given genetic and/or drug information. If response and/or adverse event markers are present, then the system 100 determines 256 the RCV given the genetic information and drug(s) received 202, 204 for the subject, as described above. The system 100 compares the RCV and MCV and assigns the greater of the two values. Accordingly, the system 100 determines 258 whether the RCV is greater than the MCV. If the MCV is greater, the system 100 assigns 254 the MCV as the score for the given genetic and/or drug information. If the RCV is greater, the system 100 assigns 260 the RCV as the score for the given genetic and/or drug information. Accordingly, the system 100 categorizes 262 the drug according to the assigned 254, 260 score.

If the system 100 does determine 218 that there are problematic drug-gene interactions for the user, the system 100 can identify 220 one or more alternative drugs to the analyzed drug. In one embodiment, the alternative drugs could be determined from the product category of the drug at issue. For example, if the drug being analyzed was a beta blocker, then the system 100 could identify 220 an alternative beta blocker for the user. As another example, if the drug being analyzed was an opioid, then the system 100 could identify 220 an alternative opioid for the user. In one embodiment, the identified 220 alternative drug could further be analyzed to identify 206 any drug-gene interactions associated with the identified 220 alternative drug, as indicated in FIG. 2. This embodiment can be beneficial because it could be desirable to check the proposed drug for any problematic gene-drug interactions so that the system 100 only proposes alternative drugs that were likewise analyzed for their gene-drug interactions. Further, the system 100 can repeat the process of identifying 206 gene-drug interactions continuously until a suitable alternative drug (i.e., a drug that does not have any problematic gene-drug interactions) that is in the same product category of the originally analyzed drug is identified.

Once the system 100 executing the process 200 has identified a suitable alternative drug, the system 100 can provide 222 the results. In one embodiment, the system 100 can provide 222 the results to the user 112. In another embodiment, the system 100 can provide 222 the results to the user's healthcare provider 114. In yet another embodiment, the system 100 could provide 222 the results to both the user 112 and the healthcare provider 114. The results can include whether any gene-drug or drug-drug interactions were identified and/or a proposed alternative drug. In one embodiment, the system 100 could provide the patient and/or third party with data, a report, or a summary indicating the pharmacokinetic impact of the patient's particular genetic mutation(s) on the medication(s) currently being taken by the patient. Such an embodiment could be beneficial in order to further educate patients in managing and taking an active role in their own healthcare. For example, FIG. 3B illustrates a reporting GUI 310 provided by the system 100. In this example, the reporting GUI 310 includes an alert 311 indicating that problematic drug-drug and/or gene-drug interaction were identified for the user. In this example, a widget 312 of the reporting GUI 310 indicates that there is a problematic drug-drug interaction between two of the drugs being taken by the user. Further, a second widget 314 of the reporting GUI 310 further explains the identified problematic gene-drug interaction. Finally, the reporting GUI 310 lists alternative drugs 316 for each of the drugs that the user is taking for which problematic gene-drug interaction and/or drug-drug interactions were identified.

Further, the process 200 illustrated in FIG. 2 can be repeated for each drug that the user is taking. The analyses for each of the drugs by the process 200 executed by the system 100 can be incorporated into the report provided 222 to the user.

It should further be noted that although the functions and/or steps of the process 200 are depicted in a particular order or arrangement, the depicted order and/or arrangement of steps and/or functions is simply provided for illustrative purposes. Unless explicitly described herein to the contrary, the various steps and/or functions of the process 200 can be performed in different orders, in parallel with each other, in an interleaved manner, and so on.

### Drug Interaction Analysis System—Application Programmable Interface

As generally described above, a variety of different external systems (e.g., third party systems) can be communicably coupled to the computer system 102 or otherwise be configured to interface with the computer system 102. In some embodiments, the external systems can be communicably coupled to the computer system 102 via an application programmable interface (API). An API is a set of protocols, routines, and tools for building software applications. Accordingly, the API defines a standard set of rules that allows external systems to communicate with the computer system 102. The API can enable external systems to exchange data with the computer system 102 in order to provide access to the drug-drug and gene-drug analyses provided by the computer system 102 (as described above) to determine drug effectiveness. The API could allow a variety of different external systems to submit queries to the computer system 102 or otherwise make use of the computer system 102 in order to instantaneously allow external systems to receive responses from the computer system 102, without the need for manual queries to be generated as typical for this field.

The API can support a variety of different endpoints, such as an API call to retrieve patient results based on a drug lookup. The API call could be structed to, for example, receive a particular drug (or set of drugs) for a subject and identifying information for the subject. In response to the call, the computer system 102 could return data indicating that the patient data was located within the internal database 108, the assigned category for the drug (i.e., whether any drug-gene and/or drug-drug interactions were identified and the severity of those interactions), and the identified drug-gene and/or drug-drug interactions.

This data could then either be presented to the user (e.g., via a graphical user interface, as shown in FIGS. 3A and 3B) or otherwise utilized by the external system (e.g., a healthcare provider system 114) initiating the query. As generally discussed above, the genetic testing data 118 for the particular subject may have been previously received by the computer system 102 from an appropriate genetic testing facility and/or otherwise uploaded to the internal database 108.

Aspects of the present invention will now be illustrated by way of example only and with reference to the following experimentation.

### Example 1—Efavirenz

Efavirenz, sold under SUSTIVA®, is a human immunodeficiency virus type 1 (HIV-1) specific, non-nucleoside, reverse transcriptase inhibitor (NNRTI). Efavirenz is metabolized by CYP3A4/5, CYP2B6, CYP2C9, and CYP2C19; accordingly, these genes are utilized to analyze whether any problematic interactions exist based on the subject's genetic test results associated with these genes. In this example, an illustrative patient has the following genetic test results: 3A4 PM, 3A5 IM, 2B6 EM, 2C9 IM, 2C19 PM, where PM, IM, and EM are metabolic phenotypes. The metabolic phenotypes are determined by (i) conversion of the mutations into associated phenotypes and (ii) categorization of the two phenotypes (one from each parent), also known as the diplotype, into a composite phenotype and associated PCD via consulting a diplotype-to-phenotype conversion table. Based on the aforementioned genetic result data, the PCD for CYP3A5 is five (i.e., “yellow”), the PCD for CYP2B6 is one (i.e., “green”), the PCD for CYP2C9 is five (i.e., “yellow”), and the PCD for CYP2C19 is ten (i.e., “red”). As noted above, a relative importance is assigned by the computer system 102 to each of the various components of the MCV calculation. These weights are set forth below. Accordingly, the MCV is calculated as follows for efavirenz given the above genetic information:

$${MCV}_{efavirenz} = ( {PCD}_{CYP 3 A 5} \times {I}_{CYP 3 A 5} ) + ( {PCD}_{CYP 2 B 6} \times {I}_{CYP 2 B 6} ) + ( {PCD}_{CYP 2 C 9} \times {I}_{CYP 2 C 9} ) + ( {PCD}_{CYP 2 C 19} \times {I}_{CYP 2 C 19} )$$

$${MCV}_{efavirenz} = ( 5 \times 0.60 ) + ( 1 \times 0.30 ) + ( 5 \times 0.05 ) + ( 10 \times 0.05 ) = 4.05$$

Therefore, for this particular patient, the MCV of efavirenz is 4.05. Accordingly, the computer system 100 would assign a yellow phenotypic color designation, indicating a moderate risk associated with this drug for this patient. As described above, this categorization could be displayed to the user via a graphical user interface.

Notably, although efavirenz is metabolized by CYP3A4 and CYP3A5, CYP3A4 is ignored by the computer system 102 because CYP3A4 and CYP3A5 are treated as a combined function because they act identically on the drugs they affect. Accordingly, the computer system 102 and processes described herein can utilize the best result between CYP3A4 and CYP3A5 for calculating the score for a drug.

### Example 2—Simvastatin

Simvastatin is used to lower low-density lipoprotein (LDL-C) cholesterol in the blood. In a patient of African descent, simvastatin is metabolized by CYP3A4/5 and is associated with an adverse effect gene (SLCO1B1); accordingly, these genes are utilized to analyze whether any problematic interactions exist based on the subject's genetic test results associated with these genes. In this example, an illustrative patient has the following genetic test results: 3A4 IM, 3A5 EM, SLCO1B1. Based on the aforementioned genetic result data, the PCD for CYP3A5 is one (i.e., “green”). As noted above, a relative importance is assigned by the computer system 102 to each of the various components of the MCV calculation. These weights are set forth below. Accordingly, the MCV is calculated as follows for simvastatin given the above genetic information:

$${MCV}_{simvastatin} = ( {PCD}_{CYP 3 A 5} \times {I}_{CYP 3 A 5} )$$

$${MCV}_{simvastatin} = ( 1 \times 1.0 ) = 1.0$$

Once again, it should be noted that although simvastatin is metabolized by both CYP3A4 and CYP3A5, only one of these two genes is analyzed for the reasons discussed above. Therefore, for this particular patient, the MCV of simvastatin is 1.0. However, because simvastatin additionally has an adverse event marker associated with it, the computer system 102 would additionally determine the RCV for simvastatin. In this case, an RCV of five (i.e., “yellow”) has been assigned to SLCO1B1 for this drug. Because the RCV is larger than the calculated MCV, the computer system 102 would assign the RCV as the drug score. Accordingly, the computer system 100 would assign a yellow phenotypic color designation, indicating a moderate risk associated with this drug for this patient.

### Example 3—Desvenlafaxine

Desvenlafaxine is a selective serotonin and norepinephrine reuptake inhibitors (SNRI) that is used to treat depression. In a patient of non-African descent, simvastatin is metabolized by CYP3A4/5 and CYP2D6; accordingly, these genes are utilized to analyze whether any problematic interactions exist based on the subject's genetic test results associated with these genes. In this example, an illustrative patient has the following genetic test results: 3A4 EM, 3A5 PM, and 2D6 EM. Based on the aforementioned genetic result data, the PCD for CYP3A5 is one (i.e., “green”). As noted above, a relative importance is assigned by the computer system 102 to each of the various components of the MCV calculation. These weights are set forth below. For some drugs, the computer system 102 can further apply a general metabolic relevance factor (MRF) to the MCV calculation where only a fraction of the relevant genes metabolize the drugs in vivo. For desvenlafaxine, only 5-10% of CYP genes metabolize the drug. Therefore, the computer system 102 can apply a general relevance factor or weight to the overall MCV calculation. Accordingly, the MCV is calculated as follows for simvastatin given the above genetic information:

$${MCV}_{desvenlafaxine} = MRF \times [ ( P C {D}_{CYP 3 A 5} \times {I}_{CYP 3 A 5} ) + ( P C {D}_{CYP 2 D 6} \times {I}_{CYP 2 D 6} ) ]$$

$${MCV}_{desvenlafaxine} = 0.1 \times [ ( 1 \times 0.9 ) + ( 1 \times 0.1 ) ] = 0.1$$

Once again, it should be noted that although simvastatin is metabolized by both CYP3A4 and CYP3A5, only one of these two genes is analyzed for the reasons discussed above. Therefore, for this particular patient, the MCV of desvenlafaxine is 0.1. Accordingly, the computer system 100 would assign a green phenotypic color designation, indicating that there is little to no risk associated with this drug for this patient.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art, all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

The functions and process steps herein may be performed automatically or wholly or partially in response to user command. An activity (including a step) performed automatically is performed in response to one or more executable instructions or device operation without user direct initiation of the activity.

1. A method for analyzing drug interactions for a user, the method comprising:
accessing a genetic test result for the user and a proposed drug for administration to the user, wherein the proposed drug is in a drug category;
determining whether the proposed drug is present in an internal database, wherein the internal database comprises a list of genes are associated with a plurality of drugs based on clinical relevance of the gene to the actions of the plurality of drugs;
in response to determining that the proposed drug is not present in the internal database:
searching an external database for a major metabolite and an enzyme responsible for forming the major metabolite, wherein the major metabolite and the enzyme are associated with the proposed drug, and
determining whether there is a mutation in a user gene associated with the enzyme that pharmacokinetically impacts the proposed drug based on the genetic test result;
in response to determining that the proposed drug is present in the internal database:
determining a clinically relevant enzyme associated with the proposed drug from the internal database, and
determining whether there is a mutation in the clinically relevant gene based on the genetic test result;
determining whether there is a problematic gene-drug interaction associated with the proposed drug by:
determining a metabolic component value for the proposed drug based on the received genetic test result,
determining a response component value for the proposed drug based on the received genetic test result,
assigning a drug category from a plurality of categories for the proposed drug based on the greater of the metabolic component value and the response component value, wherein the drug category indicates a severity of any drug-gene interactions for the user and the proposed drug, and
determining whether there is at least a threshold pharmacokinetic impact on the proposed drug caused by the mutation based on the severity of any drug-gene interactions for the user;
in response to determining that there is the problematic gene-drug interaction, identifying an alternative drug from the drug category; and
providing a report to the user, the report comprising the problematic gene-drug interaction and the alternative drug.

2. The method of claim 1, further comprising:
providing, to a healthcare provider associated with the user, the report.

3. The method of claim 1, further comprising:
determining whether the user has a problematic gene-drug interaction with the alternative drug.

4. The method of claim 1, wherein the threshold pharmacokinetic impact comprises over-metabolizing functionality of the enzyme with respect to the proposed drug.

5. The method of claim 1, wherein the threshold pharmacokinetic impact comprises under-metabolizing functionality of the enzyme with respect to the proposed drug.

6. A system for analyzing drug interactions for a user, the system comprising:
a processor; and
a memory coupled to the processor, the memory storing instructions that, when executed by the processor, cause the system to:
access a genetic test result for the user and a proposed drug for administration to the user, wherein the proposed drug is in a drug category,
determine whether the proposed drug is present in an internal database, wherein the internal database comprises a list of genes are associated with a plurality of drugs based on clinical relevance of the gene to the actions of the plurality of drugs;
in response to determining that the proposed drug is not present in the internal database:
search an external database for a major metabolite and an enzyme responsible for forming the major metabolite, wherein the major metabolite and the enzyme are associated with the proposed drug, and
determine whether there is a mutation in a user gene associated with the enzyme that pharmacokinetically impacts the proposed drug based on the genetic test result,
in response to determining that the proposed drug is present in the internal database:
determine a clinically relevant enzyme associated with the proposed drug from the internal database, and
determine whether there is a mutation in the clinically relevant gene based on the genetic test result;
determine whether there is a problematic gene-drug interaction associated with the proposed drug by:
determining a metabolic component value for the proposed drug based on the received genetic test result,
determining a response component value for the proposed drug based on the received genetic test result,
assigning a drug category from a plurality of categories for the proposed drug based on the greater of the metabolic component value and the response component value, wherein the drug category indicates a severity of any drug-gene interactions for the user and the proposed drug, and
determining whether there is at least a threshold pharmacokinetic impact on the proposed drug caused by the mutation based on the severity of any drug-gene interactions for the user;
in response to determining that there is the problematic gene-drug interaction, identify an alternative drug from the drug category; and
provide a report to the user, the report comprising the problematic gene-drug interaction and the alternative drug.

7. The system of claim 6, wherein the memory stores instructions that, when executed by the processor, cause the system to:
provide, to a healthcare provider associated with the user, the report.

8. The system of claim 6, wherein the memory stores instructions that, when executed by the processor, cause the system to determine whether the user has a problematic gene-drug interaction with the alternative drug.

9. The system of claim 6, wherein the threshold pharmacokinetic impact comprises over-metabolizing functionality of the enzyme with respect to the proposed drug.

10. The system of claim 6, wherein the threshold pharmacokinetic impact comprises under-metabolizing functionality of the enzyme with respect to the proposed drug.
